(19) United States
(12) Patent Application Publication
(10) Pub. No.: US 2026/0335654 A1
(43) Pub. Date: Oct. 29, 2026

(54) CONDUCTION SYSTEM PACING CONTROL

(71) Applicant: Medtronic, Inc., Minneapolis, MN (US)

(72) Inventor: Xiaohong Zhou, Woodbury, MN (US)

(21) Appl. No.: 19/120,156

(22) PCT Filed: Oct. 3, 2023
(86) PCT No.: PCT/IB2023/059920

§ 371 Date: Apr. 10, 2025

Related U.S. Application Data
(60) Provisional application No. 63/380,156, filed on Oct. 19, 2022.

(51) Int. Cl. A61N 1/368

(52) U.S. Cl. CPC A61N 1/3682

(57) ABSTRACT

An implantable medical device includes a first lead carrying a first set of electrodes and configured to be implanted in an inter-ventricular septum of a heart to position at least one electrode of the first set of electrodes in a left bundle branch area proximate a left-ventricular septum. The implantable medical lead further includes a second lead carrying a second set of electrodes and configured to be implanted in a coronary sinus of the heart. Sensing circuitry of the implantable medical device is configured to sense depolarizations from pacing pulses delivered by the sets of electrodes. Processing circuitry is configured to determine conduction times of the pacing pulses. The processing circuitry is configured to determine, based on the first conduction time and the second conduction time, whether a left bundle branch area pulse includes left bundle branch pacing.

This application claims the benefit of U.S. Provisional Patent Application Ser. No. 63/380,156, filed Oct. 19, 2022, the entire content of which is incorporated herein by reference.

### TECHNICAL FIELD

The disclosure relates generally to medical device systems and, more particularly, medical device systems configured for cardiac pacing.

### BACKGROUND

Some types of implantable medical devices (IMDs), such as cardiac pacemakers or implantable cardioverter defibrillators, may be used to provide cardiac therapy to a patient via one or more electrodes. The cardiac therapy may be delivered to the heart in the form of pulses or shocks for pacing, cardioversion or defibrillation, or cardiac resynchronization therapy (CRT). CRT may help enhance cardiac output by resynchronizing the electromechanical activity of the ventricles of the heart in patients with conditions such as ventricular dyssynchrony. Some IMDs may sense intrinsic depolarizations of the heart and control the delivery of CRT to the heart based on the sensed intrinsic depolarizations.

Conduction system pacing (CSP) is a technology that uses the heart's native conduction system to provide paced depolarizations and resulting contractions that better mimic intrinsic depolarizations and contractions, which may improve the health and pumping efficiency of the heart. Example types of conduction system pacing include His bundle pacing, left bundle branch pacing (LBBP), right bundle branch pacing (RBBP), and bilateral bundle branch pacing (BBBP). Example locations from which the conduction system may be accessed include the ventricular septum via the right ventricle, and the atrio-ventricular septum via the right atrium, e.g., at the area of the triangle of Koch. In some examples, CSP may provide cardiac resynchronization without requiring delivery of cardiac pacing to the left side of the heart.

### SUMMARY

CSP, such as LBBP, is a physiological pacing modality that, in some respects, may be superior to conventional biventricular CRT. For example, CSP may be more energy-efficient than conventional biventricular CRT because CSP may deliver cardiac pacing using a single ventricular lead and the heart's natural conduction system (His-purkinje fibers) to rapidly conduct an action potential down the ventricular septum, spreading the depolarization wavefront quickly through the remaining ventricular myocardium, and producing a coordinated contraction of the ventricular muscle mass. In contrast, conventional, or traditional, CRT pacing therapy may be described as delivering pacing pulses into myocardial tissue that is not part of the cardiac conduction system of the patient's heart such that, e.g., the pacing pulses trigger electrical activation that propagates primarily from one myocardial cell to another myocardial cell (also referred to as “cell-to-cell”) as opposed to propagating within the cardiac conduction system prior to the myocardial tissue. Further, conventional CRT may rely on the use of two ventricular leads for pacing therapy. However, it may be challenging to confirm successful LBB capture, and CSP more generally, during implantation and post-implantation. Unsuccessful CSP may result in myocardial pacing. For example, unsuccessful LBB capture can result in local left-ventricular septal pacing (LVSP) without a direct capture of the left bundle branch (LBB).

In general, this disclosure is directed to techniques for determining whether electrical stimulation has achieved CSP, or instead resulted in less efficacious myocardial pacing. In some examples, an implantable medical device comprises: a connector block configured to couple to a plurality of leads comprising: a first lead carrying a first set of electrodes and configured to be implanted in an inter-ventricular septum of a heart to position at least one electrode of the first set of electrodes in a left bundle branch area proximate a left-ventricular septum; and a second lead carrying a second set of electrodes and configured to be implanted in a coronary sinus of the heart; and sensing circuitry configured to: sense, via the second set of electrodes, a first depolarization resulting from a first pacing pulse delivered by the first set of electrodes; and sense, via the first set of electrodes, a second depolarization resulting from a second pacing pulse delivered by the second set of electrodes; and processing circuitry configured to: determine a first conduction time of the first pacing pulse from the left-ventricular septum to the coronary sinus based on the sensing of the first depolarization; determine a second conduction time of the second pacing pulse from the coronary sinus to the left-ventricular septum based on the sensing of the second depolarization; and determine, based on the first conduction time and the second conduction time, whether the first pacing pulse comprises left bundle branch pacing.

In some examples, a medical system comprises: a plurality of leads comprising: a first lead carrying a first set of electrodes and configured to be implanted in an inter-ventricular septum of a heart to position at least one electrode of the first set of electrodes in a left bundle branch area proximate a left-ventricular septum; and a second lead carrying a second set of electrodes and configured to be implanted in a coronary sinus of the heart; an implantable medical device coupled to the plurality of leads, the implantable medical device comprising: a connector block configured to couple to the plurality of leads; sensing circuitry configured to: sense, via the second set of electrodes, a first depolarization resulting from a first pacing pulse delivered by the first set of electrodes; and sense, via the first set of electrodes, a second depolarization resulting from a second pacing pulse delivered by the second set of electrodes; and processing circuitry configured to: determine a first conduction time of the first pacing pulse from the left-ventricular septum to the coronary sinus based on the sensing of the first depolarization; determine a second conduction time of the second pacing pulse from the coronary sinus to the left-ventricular septum based on the sensing of the second depolarization; and determine, based on the first conduction time and the second conduction time, whether the first pacing pulse comprises left bundle branch area pacing; and an external device configured to: communicatively couple to the implantable medical device; and output an indication of whether the first pacing pulse comprises left bundle branch area pacing.

In some examples, a method comprises: sensing, via a second set of electrodes of a second lead of an implantable medical device, a first depolarization resulting from a first pacing pulse delivered by a first set of electrodes of a first lead of the implantable medical device, wherein the second set of electrodes is positioned in the coronary sinus, wherein the first set of electrodes is implanted in an inter-ventricular septum of a heart, and wherein at least one electrode of the first set of electrodes in a left bundle branch area proximate a left-ventricular septum; and sensing, via the first set of electrodes, a second depolarization resulting from a second pacing pulse delivered by the second set of electrodes; determining, by processing circuitry of the implantable medical device, a first conduction time of the first pacing pulse from the left-ventricular septum to the coronary sinus based on the sensing of the first depolarization; determining, by the processing circuitry, a second conduction time of the second pacing pulse from the coronary sinus to the left-ventricular septum based on the sensing of the second depolarization; and determining, by the processing circuitry, based on the first conduction time and the second conduction time, whether the first pacing pulse comprises left bundle branch pacing.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 is a conceptual diagram illustrating an example of a medical device system including an implantable medical device and an external device in conjunction with a heart of a patient.

FIG. 2 is a conceptual diagram illustrating portions of the medical device system of FIG. 1 in conjunction with the heart of the patient.

FIG. 3 is a functional block diagram illustrating an example configuration of the example implantable medical device of FIG. 1.

FIG. 4 is a functional block diagram illustrating an example system that includes an external device, such as a server, and one or more computing devices that are coupled to the implantable medical device and the external device of FIG. 1 via a network.

FIGS. 5A-5B are timing diagrams illustrating sensing of ventricular activation.

FIG. 6 is a flow diagram illustrating an example technique for selecting between conduction system pacing and cardiac resynchronization pacing in accordance with techniques of this disclosure.

### DETAILED DESCRIPTION

In general, this disclosure describes example techniques related to conduction system pacing (CSP). CSP is a technique in which one or more pacemaker devices use the heart's native electrical conduction system to conduct electrical signals that cause depolarization of heart muscles, which ultimately causes synchronous contraction of the ventricles. In this manner, CSP may provide benefits associated with cardiac resynchronization therapy (CRT). A system may provide different types of CSP depending upon which portion of the heart's native electrical conduction system are targeted with electrical stimulation. Examples of CSP include left bundle branch pacing (LBBP), right bundle branch pacing (RBBP), bilateral bundle branch pacing (BBBP), His bundle pacing (HBP), and the like.

As used herein, left bundle branch area pacing (LBBAP) refers to pacing near the LBB and may include left bundle branch pacing (LBBP) with LBB capture and left ventricular septal pacing (LVSP) without LBB capture. LBBP may be preferable to LVSP because LBBP captures part of the heart's natural conduction system while LVSP does not. As used herein, LBB refers to the left bundle branches located in the sub-endocardial region of the left-ventricular septum. The ventricular septum (e.g., the inter-ventricular septum) can be divided into the right-ventricular septum on the right side of the ventricular septum and the left-ventricular septum on the left side of the ventricular septum.

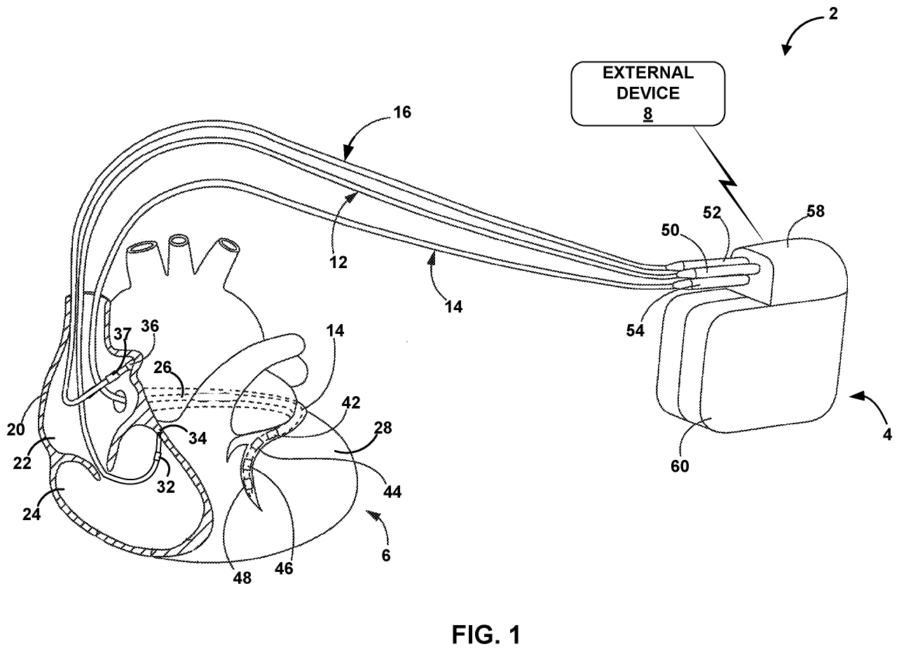

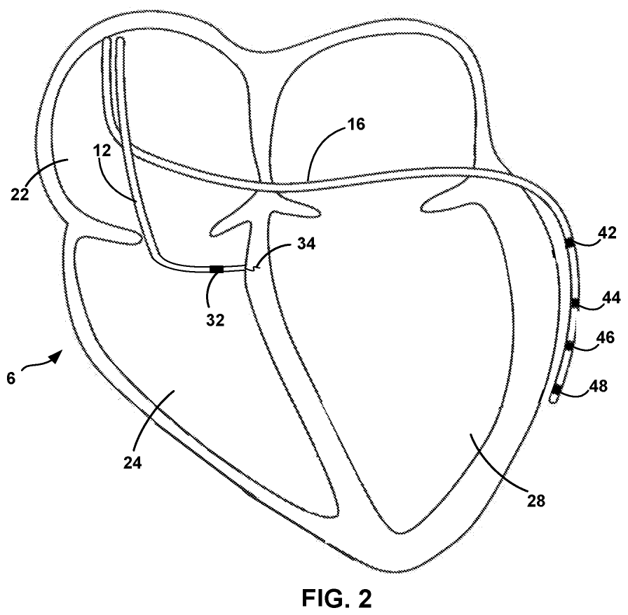

FIG. 1 is a conceptual diagram illustrating an example of a medical device system 2 including an implantable medical device 4 (“IMD 4”) and an external device 8 in conjunction with a heart 6 of a patient. FIG. 2 is a conceptual diagram further illustrating portions of medical device system 2 in conjunction with heart 6. Medical device system 2 is an example of a medical device system configured to implement the example techniques described herein for determining whether a pacing pulse includes LBBP and for selecting between CSP and biventricular CRT pacing in accordance with techniques of this disclosure.

In some examples, IMD 4 may be an implanted, multi-channel cardiac pacemaker, implantable cardioverter-defibrillator (ICD), implantable pulse generator (IPG), leadless (e.g., intracardiac) pacemaker, extravascular pacemaker and/or ICD, or other IMD or combination of such IMDs configured to deliver CSP to heart 6. In some examples, IMD 4 may be configured to sense electrical signals corresponding to the depolarization and repolarization of heart 6, e.g., a cardiac electrogram (EGM), via electrodes on one or more leads 12, 14, and 16 or the housing of IMD 4. Additionally, or alternatively, IMD 4 may sense electrical signals corresponding to the depolarization and repolarization of heart 6 via extravascular electrodes (e.g., electrodes positioned outside the vasculature of the patient), such as epicardial electrodes, external surface electrodes, subcutaneous electrodes, and the like. In any such examples, the configurations of electrodes used by IMD 4 for sensing and pacing may be unipolar or bipolar. In some examples, IMD 4 may determine heart rate to, e.g., detect arrhythmia, based on the electrical signals sensed via the electrodes. IMD 4 may also deliver therapy in the form of electrical signals to heart 6 via electrodes located on one or more leads 12, 14, and 16 or a housing of IMD 4. In the illustrated example, IMD 4 is connected to leads 12, 14 and 16, and may be communicatively coupled to external device 8.

Leads 12, 14, and 16 extend into heart 6 of the patient to sense electrical activity of heart 6 and to deliver electrical stimulation to heart 6. In the example shown in FIG. 1, first lead 12 extends through one or more veins (not shown), vena cava 20, right atrium 22 (“RA 22”), right ventricle 24 (“RV 24”), and into the inter-ventricular septum for sensing cardiac signals and delivering CSP, e.g., LBBP. First lead 12 may carry a first set of electrodes. Second lead 14 may carry a second set of electrodes. In examples where IMD 4 includes a third lead 16, third lead 16 may carry a third set of electrodes. As used herein, a set may refer to one or more elements. For example, a set of electrodes may refer to one or more electrodes.

First lead 12 may be configured to be implanted in the RV of heart 6 to position at least one electrode of the first set of electrodes in a left bundle branch area proximate a left-ventricular septum. In the example of FIG. 1, a distal end of first lead 12 is positioned at the inter-ventricular septum between RV 24 and left ventricle 28 (“LV 28”) via RV 24 for delivery of CSP. Electrode 34 of first lead 12 may extend into the inter-ventricular septum to facilitate capture of the conduction system with electrical stimulation delivered via electrode 34. An electrode used to deliver CSP may be positioned in other locations, such as the atrioventricular septum via the triangle of Koch, in other examples. In some examples, system 2 may include one or more leadless pacing devices configured to deliver CSP, e.g., instead of one or more of leads 12, 14, and 16.

Second lead 14 may be configured to be implanted in the coronary sinus (CS) of heart 6 to position the second set of electrodes in the CS. In the example of FIG. 1, second lead 14 extends through one or more veins, vena cava 20, RA 22, and into coronary sinus 26 (illustrated in phantom) to a region adjacent to the free wall of LV 28 of heart 6 for sensing left-ventricular cardiac signals and delivering therapeutic signals to LV 28. In some examples, second lead 14 may also be referred to as a CS lead. As further shown in the example of FIG. 1, third lead 16 extends through one or more veins and vena cava 20 and is positioned such that a distal end of third lead 16 is in the vicinity of RA 22 and vena cava 20 for sensing right atrial cardiac signals and delivering therapeutic signals to RA 22.

In the illustrated example, first lead 12 includes bipolar electrodes 32 and 34, which may be located adjacent to a distal end of first lead 12. Third lead 16 includes bipolar electrodes 36 and 37, which may be located adjacent to a distal end of third lead 16. Second lead 14 may be a multipolar LV lead and may include electrodes 42, 44, 46, and 48. In some examples, electrodes 42, 44, 46, and 48 may be located adjacent to a distal end of second lead 14, as illustrated in FIGS. 1 and 2.

Electrodes 34 and/or 36 may be fixed or extendable helix tip electrodes and may be mounted on respective insulative electrode heads. Electrodes 32 and 37 may be ring electrodes forming an outer surface of the leads 12 and 16 respectively. One example of such a lead can be the SELECTSECURE™ 3830 lead by Medtronic. Further, while electrodes 32 and 37 are generally shown as being withing the chamber volume of the RV and RA, in other examples, electrodes 32 and 37 may be positioned within a portion of the cardiac tissue. For example, electrode 32 may be positioned within the inter-ventricular septum proximate the right-ventricular septum and right bundled branch. Electrode 32 may be used in such examples to facilitate capture of the conduction system (e.g., right bundle branch fibers).

In some examples, electrodes 32-48 of leads 12, 14, and 16 may be electrically coupled to a respective conductor within a lead body of a corresponding one of leads 12, 14, and 16, and thereby coupled to circuitry within IMD 4. In some examples, leads 12, 14, and 16 respectively include in-line connectors 50, 52, and 54. IMD 4 may further include a connector block 58 and a hermetically-sealed housing 60. In-line connectors 50, 52, and 54 may be configured to fit into corresponding bipolar bores of connector block 58, which may be coupled to electrically insulated conductors within leads 12, 14, and 16, thereby connecting electrodes 32-48 to IMD 4. In this way, connector block 58 may be configured to couple to leads 12, 14, and 16.

In some examples, one or more outward-facing portions of housing 60 may be uninsulated, and thus may enable housing 60 to be used as a housing electrode. In some examples, substantially all of housing 60 may be uninsulated, such that substantially all of housing 60 defines the housing electrode. In some other examples, housing 60 may define one or more additional housing electrodes (not shown), which may be defined by corresponding divisions between insulated and uninsulated portions of housing 60. In some examples, IMD 4 may be configured for bipolar sensing of electrical signals corresponding to a cardiac electrogram of heart 6 via any bipolar combination of electrodes 32-48. In other examples, IMD 4 may be configured for unipolar sensing of electrical signals corresponding to a cardiac electrogram of heart 6 via any one of electrodes 32-48 in combination with housing electrode 60.

In some examples, medical device system 2 may include extravascular electrodes, such as subcutaneous electrodes, substernal electrodes, epicardial electrodes, and/or patch electrodes, instead of or in addition to the electrodes of leads 12, 14, and 16 illustrated in FIG. 1. In some other examples, a medical device configured to deliver cardiac therapy may not necessarily be implanted in the patient. In some such examples, a medical device may deliver pacing and other therapies to heart 6 via percutaneous leads that extend through the skin of the patient to one or more locations within or outside of heart 6.

In some other examples, medical device system 2 may include any suitable number of leads coupled to IMD 4 and extending to any suitable location within or proximate to heart 6. For example, medical device system 2 may include a dual-chamber IMD instead of a three-chamber IMD, such as IMD 4. In one example of a dual chamber configuration, IMD 4 is connected to leads 12 and 16.

Instead of or in addition to IMD 4, medical device system 2 may include one or more leadless (e.g., intracardiac) pacing devices (LPDs). In such examples, the one or more LPDs may include therapy delivery circuitry and processing circuitry within a housing configured for implantation on or within one of the chambers of heart 6. In such systems, the one or more pacing devices, which may include one or more LPDs and/or an IMD coupled to one or more leads, may communicate to coordinate sensing and pacing in various chambers of heart 6 to provide CSP and CRT according to the techniques described herein.

External device 8 may be a computing device (e.g., used in a home, ambulatory, clinic, or hospital setting) to communicate with ICM 10 via wireless telemetry. External device 8 may include or be coupled to a remote patient monitoring system, such as Carelink®, available from Medtronic plc, of Dublin, Ireland. External device 8 may be, as an example, a programmer, external monitor, or a consumer device (e.g., tablet or smart phone). In some examples, external device 8 may receive data, alerts, patient physiological information, or other information from IMD 4.

In some examples, external device 8 may be used to program commands or operating parameters into IMD 4 for controlling its functioning (e.g., when configured as a programmer for IMD 4). External device 8 may be used to interrogate IMD 4 to retrieve data, including device operational data as well as physiological data accumulated in IMD memory. The interrogation may be automatic, such as according to a schedule, or in response to a remote or local user command. Programmers, external monitors, and consumer devices are examples of external devices 18 that may be used to interrogate IMD 4. Examples of communication techniques used by IMD 4 and external device 8 include radiofrequency (RF) telemetry, which may be an RF link established via Bluetooth, WiFi, or medical implant communication service (MICS). In some examples, external device 8 includes processing circuitry. The processing circuitry of external device 8 may be configured to perform any of the techniques described with respect to processing circuitry of medical device system 2, such as described further herein.

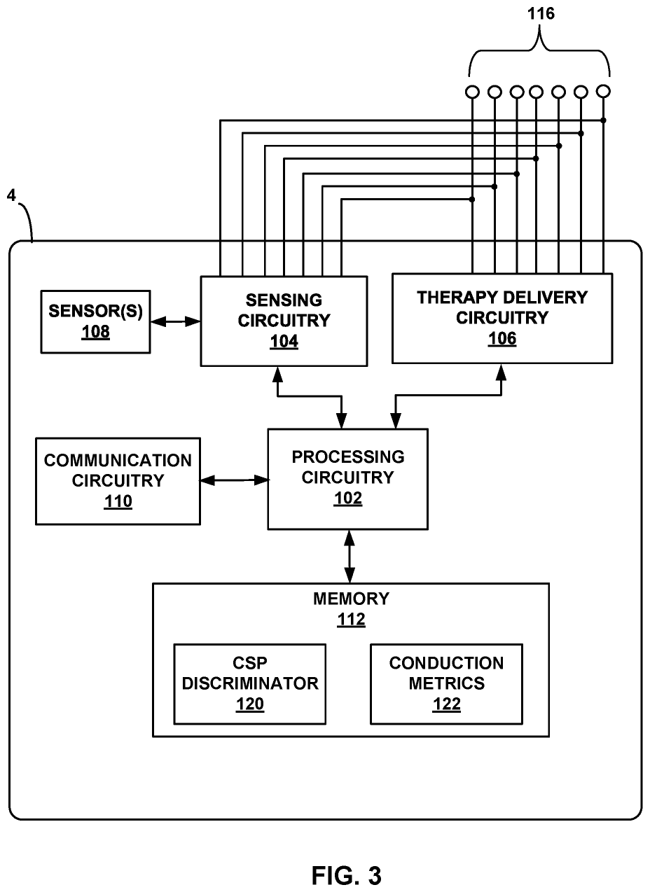

FIG. 3 is a functional block diagram illustrating an example configuration of IMD 4. As shown in FIG. 3, IMD 4 includes processing circuitry 102, sensing circuitry 104, therapy delivery circuitry 106, sensors 108, communication circuitry 110, and memory 112. In addition, IMD 4 is coupled to one or more electrodes 116, which may be any one or more of the previously-described electrodes of medical system 2, and one or more of which may be disposed on housing 60 of IMD 4 or carried by one or more of leads 12, 14, and/or 16 connected to IMD 4. In some examples, memory 112 includes computer-readable instructions that, when executed by processing circuitry 102, cause IMD 4 and processing circuitry 102 to perform various functions attributed to IMD 4 and processing circuitry 102 herein. Memory 112 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 102 may include fixed function circuitry and/or programmable processing circuitry. Processing circuitry 102 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 102 may include multiple components, such as any combination of one or more microprocessors, one or more controllers, one or more DSPs, one or more ASICs, or one or more FPGAs, as well as other discrete or integrated logic circuitry. The functions attributed to processing circuitry 102 herein may be embodied as software, firmware, hardware or any combination thereof.

Sensing circuitry 104 and therapy delivery circuitry 106 may be selectively coupled to electrodes 116, e.g., via switching circuitry (not shown) as controlled by processing circuitry 102. The switching circuitry may include one or more transistors or other circuitry for selectively coupling electrodes 116 to circuitry of IMD 4. Sensing circuitry 104 may monitor signals from electrodes 116, e.g., intracardiac electrograms (EGMs) in order to monitor electrical activity of heart (e.g., to detect depolarizations for heart rate determination and/or to sense LV activations for determining whether electrical stimulation resulted in CSP). Sensing circuity 104 may also monitor signals from one or more other sensor(s) 108, such as to determine an activity level or activity of the patient. In some examples, sensors 108 may be one or more accelerometers e.g., one or more three-axis accelerometers), one or more temperature sensors, or one or more other sensors configured to sense physical parameters of the patient. Signals generated by such sensors may be indicative of physical parameters of the patient, such as gross body movement, posture, exertion, temperature, activity level, or other physical parameters. Sensing circuitry 104 may monitor signals from electrodes 116 and sensors 108. In some examples, sensing circuitry 104 may include one or more filters and amplifiers for filtering and amplifying signals received from one or more of electrodes 116 and/or the one or more of sensor(s) 108. Sensing circuitry 104 may also include rectification circuitry, sample-and-hold circuitry, one or more comparators, and/or analog-to-digital conversion circuitry. The functionality provided by such circuitry may be applied to the signal in the analog or digital domain.

Therapy delivery circuitry 106 may include circuitry for generating a signal, such as one or more capacitors, charge pumps, and/or current sources, as well as circuitry for selectively coupling the signal to electrodes 116, e.g., transistors or other switching circuitry. Therapy delivery circuitry 106 may be configured to deliver pacing pulses or other therapeutic stimulation signals. As will be described in greater detail below, processing circuitry 102 may be configured to control therapy delivery circuitry 106 to deliver CSP and/or CRT via selected combinations of electrodes 116.

Communication circuitry 110 may include any suitable hardware, firmware, software, or any combination thereof for communicating with another device, such as external device 8, or another IMD or sensor. For example, communication circuitry 110 may include voltage regulators, current generators, oscillators, or circuitry for generating a signal, resistors, capacitors, inductors, and other filtering circuitry for processing received signal, as well as circuitry for modulating and/or demodulating a signal according to a communication protocol. Communication circuitry 110 may also include transistors or other switching circuitry for selectively coupling transmitted signal to or receiving signals from an antenna of IMD 4 (not shown) or electrodes 116. Under the control of processing circuitry 102, communication circuitry 110 may receive downlink telemetry from, as well as send uplink telemetry to, external device 8 or another device. The patient, a clinician, or another user may retrieve data from IMD 4 using external device 8, or by using another local or networked computing device (e.g., a remote computer located with the clinician) configured to communicate with processing circuitry 102 via communication circuitry 110. In some examples, the clinician may also program parameters of IMD 4 using external device 8.

IMD 4 is an example of a device configured to determine whether electrical stimulation has achieved CSP, e.g., LBBP, or instead resulted in less efficacious myocardial pacing, e.g., LVSP. Therapy delivery circuitry 106 is configured to deliver electrical stimulation configured to provide LBBAP, e.g., via electrode 34 (FIGS. 1 and 2). Processing circuitry 102 is configured to execute CSP discriminator 120 to determine whether the electrical stimulation resulted in CSP or myocardial pacing.

Processing circuitry 102 is configured to control sensing circuitry 104 to sense one or more EGMs and ventricular activations (e.g., left-ventricular activations, left-ventricular septal activations, etc.) resulting from the electrical stimulation, via one or more of electrodes 116. Processing circuitry 102 is configured to determine one or more conduction metrics 122 based on the sensed one or more ventricular activations. Processing circuitry 102 determines whether the electrical stimulation provided CSP, e.g., LBBP, based on the one or more conduction metrics.

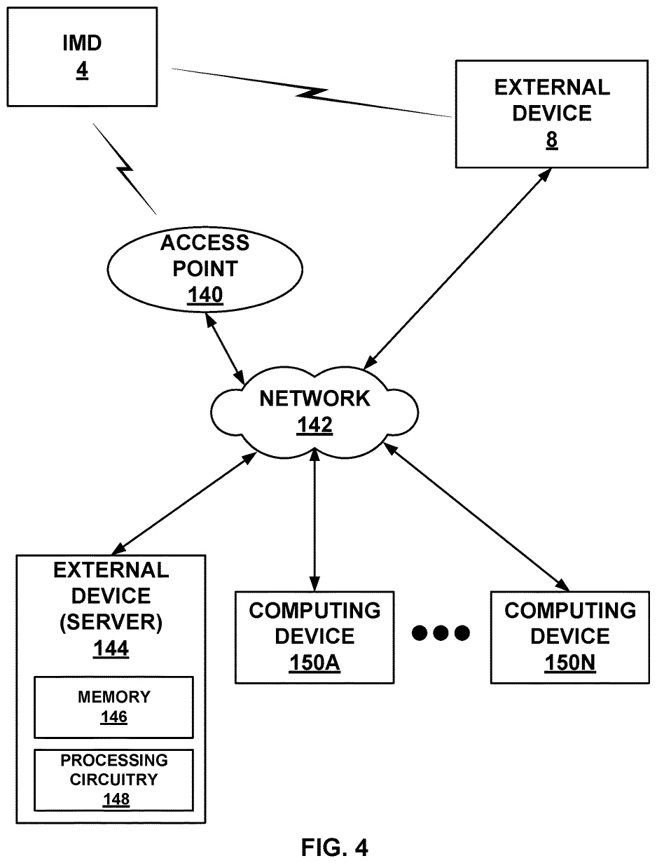

FIG. 4 is a functional block diagram illustrating an example system that includes an access point 140, a network 142, external computing devices, such as an external device (server) 144, which may include a memory 146 and/or processing circuitry 148, and one or more other computing devices 150A-150N, which may be coupled to IMD 4 and external device 8 via network 142. In this example, IMD 4 may use communication circuitry 110 to communicate with external device 8 via a first wireless connection, and to communicate with an access point 140 via a second wireless connection. In the example of FIG. 4, access point 140, external device 8, server 144, and computing devices 150A-150N are interconnected and may communicate with each other through network 142.

Access point 140 may comprise a device that connects to network 142 via any of a variety of connections, such as telephone dial-up, digital subscriber line (DSL), or cable modem, or other suitable connections. In other examples, access point 140 may be coupled to network 142 through different forms of connections, including wired or wireless connections. In some examples, access point 140 may be a user device, such as a tablet or smartphone, that may be co-located with the patient. As discussed above, IMD 4 may be configured to transmit data, such as current values and heart failure statuses, to external device 8. In addition, access point 140 may interrogate IMD 4, such as periodically or in response to a command from the patient, a clinician, or network 142, in order to retrieve data pertaining to one or more of patient parameters, delivery of therapy, or other information stored in memory 112 (FIG. 3) of IMD 4. Access point 140 may then communicate the retrieved data to server 144 via network 142.

In some cases, memory 146 of server 144 may be configured to provide a secure storage site for data collected from IMD 4 and/or external device 8. In some cases, server 144 may assemble data in web pages or other documents for viewing by trained professionals, such as clinicians, via computing devices 150A-150N. One or more aspects of the illustrated system of FIG. 4 may be implemented with general network technology and functionality, which may include or be similar to that provided by the Medtronic CareLink® Network developed by Medtronic plc, of Dublin, Ireland. In some examples, such network technology and functionality may enhance the security of the communications transmitted between the components of FIG. 4, such as the communications transmitted from external device 8 to IMD 4. For example, the network technology and functionality may validate a communication, such as patient or clinician input, transmitted from a device purporting to be external device 8 and directed toward IMD 4, by confirming the identity of the device purporting to be external device 8. In other examples, the network technology and functionality similarly may validate a communication transmitted from another device, such as a device purporting to be one or more of computing devices 150A-150N (e.g., a purported remoter computer located with a clinician) toward IMD 4. In some examples, such security features may protect the cardiac pacing delivered by IMD 4 to the patient from being disrupted, hacked, or otherwise altered by communications originating from unauthorized sources. In some examples, one or more of computing devices 150A-150N (e.g., device 150A) may be a remote computer, such as a smartphone, tablet or other smart device located with a clinician, by which the clinician may program, receive alerts from, and/or interrogate IMD 4.

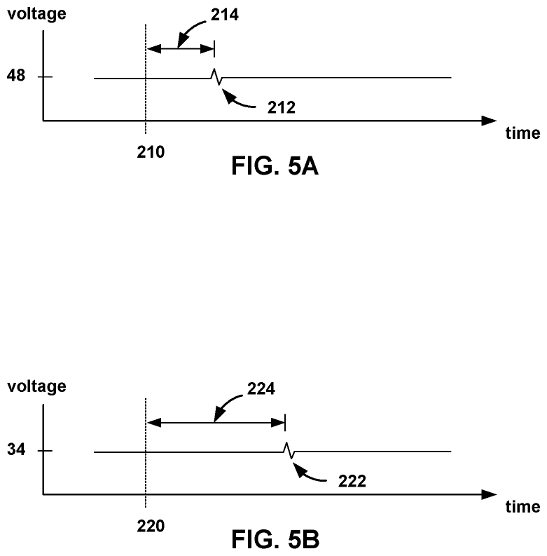

FIGS. 5A-5B are conceptual diagrams illustrating sensing of ventricular activations by sensing circuitry 104. FIG. 5A illustrates a sensed left-ventricular activation 212 detected by an electrode of second lead 14 (e.g., electrode 48) in response to a first pulse 210 delivered, for example, via electrode 34 of first lead 12. In the illustrated example, first pulse 210 is an LBBAP pulse (e.g., LBBP pulse or LVSP pulse). As shown in FIG. 5A, first pulse 210 may progress either along the His-Purkinje fibers of the LBB (e.g., an LBBP pulse) to quickly activate LV 28 and reach electrode 48, or first pulse 210 may progress along the interventricular septal wall via myocardial tissue more slowly to activate LV 28 and reach electrode 48. In either case, the time from first pacing pulse 210 to the point of detection by second lead 14 is represented by first conduction time 224.

FIG. 5B illustrates a left-ventricular activation 222 detected by an electrode of first lead 12 (e.g., electrode 34) in response to a second pulse 220 delivered, for example, via electrode 48 of second lead 14. As shown in FIG. 5B, second pulse 220 may progress more slowly via myocardial tissue from the LV free wall of the CS to the LBB area and reach electrode 34 after second conduction time 224.

In accordance with techniques of this disclosure, sensing circuitry 104 may be configured to sense, via one or more electrode of the second set of electrodes carried by second lead 14, a first depolarization resulting from a first pacing pulse delivered by one or more electrodes of the first set of electrodes carried by first lead 12. For example, sensing circuitry 104 may be configured to sense, via electrode 48, left-ventricular activation 212 resulting from first pulse 210 delivered by electrode 34. Electrode 34 may deliver first pulse 210 proximate the LBB, and electrode 48 may sense left-ventricular activation 212 at the CS after a first conduction time 214. Additionally, sensing circuitry 104 may be configured to sense, via one or more electrodes of the first set of electrodes carried by first lead 12, a second depolarization resulting from a second pacing pulse delivered by one or more electrodes of the second set of electrodes carried by second lead 14. For example, sensing circuitry 104 may be configured to sense, via electrode 34, left-ventricular activation 222 resulting from second pulse 220 delivered by electrode 48. Electrode 48 may deliver second pulse 220 to the LV at the CS, and electrode 34 may sense left-ventricular septal activation 222 at the LBB after second conduction time 224.

Processing circuitry 102 may be configured to execute CSP discriminator 120 to determine whether the electrical stimulation resulted in CSP or myocardial pacing. For example, CSP discriminator 120 may determine first conduction time 214 of the first pacing pulse from the LBB area (e.g., the left-ventricular septum, the septal myocardium, etc.) to the LV proximate the CS based on the sensing of the first depolarization. As used herein, conduction time may refer to the interval of time for a signal, such as a pacing pulse, to conduct (e.g., progress) from a first location (e.g., the LBB) in heart 6 to a second location (e.g., the CS) in heart 6. For example, CSP discriminator 120 may determine first conduction time 214 by calculating the interval of time from electrode 34 delivering first pulse 210 at the LBB to electrode 48 sensing left-ventricular activation 212 at the CS.

Additionally, CSP discriminator 120 may determine second conduction time 224 of the second pacing pulse from the CS to the LBB based on the sensing of the second depolarization. For example, CSP discriminator 120 may determine second conduction time 214 by calculating the interval of time from electrode 48 delivering second pulse 220 at the CS to electrode 34 sensing left-ventricular activation 222 at the LBB.

CSP discriminator 120 may determine whether a LBBAP pulse (e.g., the first pacing pulse) includes successful LBBP instead of myocardial capture (e.g., LVSP) based on first conduction time 214 and second conduction time 224. In some examples, CSP discriminator 120 may determine whether the LBBAP pulse includes LBB capture based on the difference between second conduction time 224 minus first conduction time 214 being greater than or equal to a conduction time differential threshold, which may be represented by the equation T₂−T₁≥x, where T₁ is first conduction time 214, where T₂ is second conduction time 224, and where x is a conduction time differential threshold (e.g., 15 milliseconds (ms), 20 ms, 25 ms, etc.). For example, CSP discriminator 120 may determine that the LBBAP pulse includes LBBP when the equation T₂-T₁≥x is true; conversely, CSP discriminator 120 may determine that the LBBAP pulse does not include LBBP (and instead includes myocardial pacing, such as LVSP) when the equation T₂-T₁≥x is false.

Processing circuitry 102 may perform one or more operations in response to CSP discriminator 120 determining whether the described CSP discrimination detects LBB capture. For example, responsive to CSP discriminator 120 determining that electrode 34 provides LBBP, processing circuitry 102 may further configure delivery of CSP by determining an atrioventricular (AV) delay length based on first conduction time 214 or other measures or cardiac synchrony or performance, such QRS width or other QRS morphological measures. For example, processing circuitry 102 may iteratively select AV delay lengths from a range of AV delay lengths, deliver CSP using the selected AV lengths, and determine corresponding first conduction times. Processing circuitry 102 may then determine the AV delay length that resulted in a first conduction time that was closest to a preferred value or shortest and use that AV delay length for CSP. In other words, processing circuitry 102 may determine a respective first conduction time of a plurality of first conduction times for each AV delay of a plurality of AV delays, and select one of the AV delays based on the plurality of first conduction times.

Additionally or alternatively, processing circuitry 102 may determine an AV delay length based on QRS width (or other measures, such as QRS morphological measures). For example, processing circuitry 102 may iteratively select AV delay lengths from a range of AV delay lengths, deliver CSP using the selected AV lengths, and determine corresponding QRS widths. Processing circuitry 102 may select the AV delay length that corresponded to the shortest QRS width. Processing circuitry 102 may follow a similar procedure for other morphological metrics.

In another example, responsive to CSP discriminator 120 determining that the LBBAP pulse does not suggest LBBP, processing circuitry 102 may be configured to confirm the determination by determining whether first conduction time 214 is greater than or equal to a first conduction time threshold (e.g., 75 ms, 80 ms, 85 ms, etc.). A determination that first conduction time 214 is greater than or equal to the first conduction time threshold may confirm that the LBBAP pulse does not include LBBP and that CSP therapy is not occurring. Consequently, responsive to CSP discriminator 120 determining that first conduction time 214 is greater than or equal to the first conduction time threshold, processing circuitry 102 may be configured to control delivery of biventricular CRT pacing via the first set of electrodes and the second set of electrodes. For example, processing circuitry 102 may be configured to cause the first set of electrodes of first lead 12 and the second set of electrodes of second lead 14 to deliver biventricular CRT pacing, such as left bundle branch-optimized cardiac resynchronization therapy (LOT-CRT). LOT-CRT may include LVSP and LV-CS pacing.

Biventricular CRT may include delaying a pacing pulse delivered via the second set of electrodes of second lead 14 relative to a pacing pulse delivered via the first set of electrodes of first lead 12 by a left-ventricular pacing pulse delay length. In some examples, processing circuitry 102 may be configured to determine the left-ventricular pacing pulse delay length based on an equation 1.5*y−T₁=z, where y is a reference conduction time (e.g., 80 ms, 85 ms, 90 ms, etc.) representing a theoretical conduction time from the LBB (e.g., the left-ventricular septum) to the CS, where T₁ is the first conduction time, and where z is the left-ventricular pacing pulse delay length. This equation may reduce or eliminate ventricular dyssynchrony (e.g., a difference in the timing, or lack of synchrony, of contractions in different ventricles in heart 6). The reference conduction time may be a pre-determined value (e.g., determined from empirical data) indicative of a typical (e.g., average, normal, common, etc.) interval of time for a signal to conduct from the LBB to the CS via the heart's native conduction system. In some examples, the left-ventricular pacing pulse delay length z may be set to a value between about ½T₁ and ½y. In any case, delaying the left-ventricular pacing pulse delay length in accordance with techniques of this disclosure may advantageously reduce or eliminate ventricular dyssynchrony, thereby improving patient outcomes.

Conversely, a determination that first conduction time 214 is less than the first conduction time threshold may indicate that the earlier determination that the LBBAP pulse does not include LBBP is false and that CSP capture is occurring. Consequently, in some examples, responsive to CSP discriminator 120 determining that first conduction time 214 is less than the first conduction time threshold, processing circuitry 102 may be configured to determine an AV delay length based on first conduction time 214 as described above to provide CSP.

It should be understood that the positions of the first set of electrodes and the second set of electrodes may affect first conduction time 214 and second conduction time 224. As such, deviations from the values described above relating to equations, thresholds, etc., attributable to variations in the positions of the first set of electrodes and the second set of electrodes are contemplated by this disclosure.

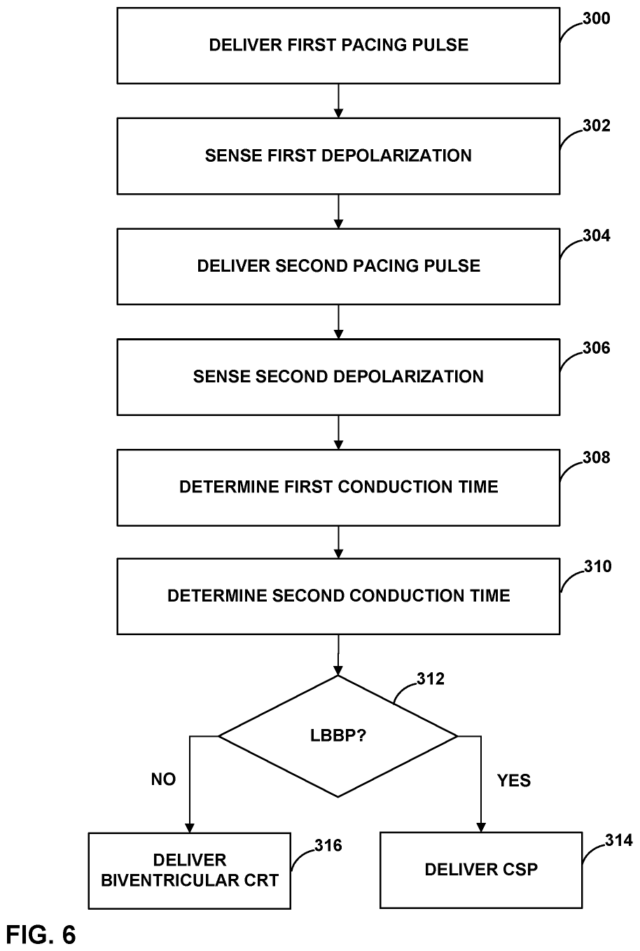

FIG. 6 is a flow diagram illustrating an example technique for differentiating conduction system and myocardial pacing in accordance with techniques of this disclosure. The example technique of FIG. 6 is described as being performed by medical device system 2 including IMD 4. In some examples, the technique of FIG. 6 may be performed by other systems including other devices. For example, the techniques of FIG. 6 may be performed by an external diagnostic device, such as a pacing system analyzer (PSA) coupled to leads 12, 14, 16 during their implantation and prior to their being coupled to IMD 4.

IMD 4 may deliver a first pacing pulse via a first set of electrodes of first lead 12 (300). For example, processing circuitry 102 may control therapy delivery circuitry 106 to deliver an electrical stimulation (e.g., first pulse 210) via electrode 34 at the LBB. Processing circuitry 102 may control sensing circuitry 104 to sense a first depolarization resulting from the first pacing pulse via a second set of electrodes of second lead 14 (302). For example, sensing circuitry 104 may sense, via electrode 48 positioned at the CS, left-ventricular activation 212 resulting from first pulse 210.

IMD 4 may deliver a second pacing pulse via the second set of electrodes of second lead 14 (304). For example, processing circuitry 102 may control therapy delivery circuitry 106 to deliver an electrical stimulation (e.g., second pulse 220) via electrode 48 at the CS. Processing circuitry 102 may control sensing circuitry 104 to sense a second depolarization resulting from the second pacing pulse via the first set of electrodes of first lead 12 (306). For example, sensing circuitry 104 may sense, via electrode 34 positioned at the LBB, left-ventricular septal activation 222 resulting from second pulse 220.

CSP discriminator 120 may determine first conduction time 214 of the first pacing pulse from the LBB to the CS based on the sensing of the first depolarization (308). For example, CSP discriminator 120 may determine first conduction time 214 by calculating the interval of time from electrode 34 delivering first pulse 210 at the LBB to electrode 48 sensing left-ventricular activation 212 at the CS.

CSP discriminator 120 may determine second conduction time 224 of the second pacing pulse from the CS to the LBB area based on the sensing of the second depolarization (310). For example, CSP discriminator 120 may determine second conduction time 214 by calculating the interval of time from electrode 48 delivering second pulse 220 at the CS to electrode 34 sensing left-ventricular septal activation 222 at the LBB.

CSP discriminator 120 may determine whether the LBBAP pulse includes successful LBBP based on first conduction time 214 and second conduction time 224 (312). In some examples, CSP discriminator 120 may determine whether the LBBAP pulse includes LBB capture based on the equation T₂−T₁≥x, where T₁ is first conduction time 214, where T₂ is second conduction time 224, and where x is a conduction time differential threshold (e.g., 15 milliseconds (ms), 20 ms, 25 ms, etc.). For example, CSP discriminator 120 may determine that the LBBAP pulse includes LBBP when the equation T₂−T₁≥x is true; conversely, CSP discriminator 120 may determine that the LBBAP pulse does not include LBB capture (and instead includes myocardial pacing, such as LVSP) when the equation T₂−T₁≥x is false.

Responsive to CSP discriminator 120 determining that the LBBAP pulse includes LBBP (“YES” block of 312), processing circuitry 102 may cause therapy delivery circuitry 106 to deliver CSP (314). In some examples, processing circuitry 102 may optimize delivery of CSP by determining an AV delay length based on first conduction time 214. For example, processing circuitry 102 may iteratively select AV delay lengths from a range of AV delay lengths and determine corresponding first conduction times. Processing circuitry 102 may then determine the AV delay length that resulted in a first conduction time that was closest to a preferred value and use that AV delay length for CSP. Additionally or alternatively, processing circuitry 102 may determine an AV delay length based on QRS width (or other morphological metric).

Responsive to CSP discriminator 120 determining that the LBBAP pulse does not include LBB capture (“NO” block of 312), processing circuitry 102 may cause therapy delivery circuitry 106 to deliver biventricular CRT (316). In some examples, processing circuitry 102 may determine a left-ventricular pacing pulse delay length for biventricular CRT based on an equation 1.5*y−T₁=z, where y is a reference conduction time (e.g., 80 ms, 85 ms, 90 ms, etc.) from the LBB to the CS, where T₁ is the first conduction time, and where z is the left-ventricular pacing pulse delay length. This equation may reduce or eliminate ventricular dyssynchrony, which may improve patient outcomes.

In some examples, subsequent to CSP discriminator 120 determining that the LBBAP pulse does not include LBBP (“NO” block of 312) and prior to delivering biventricular CRT (316), CSP discriminator 120 may confirm the determination that the LBBAP pulse does not include LBBP by determining whether first conduction time 214 is greater than or equal to a first conduction time threshold (e.g., 75 ms, 80 ms, 85 ms, etc.). A determination that first conduction time 214 is greater than or equal to the first conduction time threshold may confirm that the LBBAP pulse does not include LBBP and that CSP is not occurring. Consequently, responsive to CSP discriminator 120 determining that first conduction time 214 is greater than or equal to the first conduction time threshold, processing circuitry 102 may cause therapy delivery circuitry 106 to deliver CRT (316). Conversely, a determination that first conduction time 214 is less than the first conduction time threshold may indicate that the earlier determination that the LBBAP pulse does not include LBBP is false and that CSP is occurring. Consequently, responsive to CSP discriminator 120 determining that first conduction time 214 is less than the first conduction time threshold, processing circuitry 102 may cause therapy delivery circuitry 106 to deliver CSP (314).

Various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the terms “processor” or “processing circuitry” as used herein may refer to one or more of any of the foregoing structures or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Furthermore, the functions and techniques described in this disclosure may be provided by a medical device system that includes a plurality of IMDs. In some such examples, an IMD that may be controlled by processing circuitry to deliver ventricular pacing may not include the sensing electrodes by which the processing circuitry acquires electrograms. For example, some such medical device systems may include a leaded IMD that includes one or more intravascular leads or an extravascular ICD may include electrodes that form the first and second electrode vectors in combination with an LPD configured to be placed on or within the left ventricle and deliver ventricular pacing thereto.

In some such examples, processing circuitry of the medical device system (e.g., processing circuitry of the leaded IMD or extravascular IMD) may control the LPD to deliver ventricular pacing at a series of A-LV delays. The leaded IMD or extravascular IMD may detect pacing pulses delivered by LPD and the resulting ventricular activation in electrodes acquired by the processing circuitry from the first and second electrode vectors. The processing circuitry then may determine an updated value of a CRT parameter according to the techniques described herein and control the LPD to deliver LV pacing at the updated value of the CRT parameter to provide CRT.

Various aspects of the disclosure have been described. These and other aspects are within the scope of the following claims.

The following examples are a non-limiting list of clauses in accordance with one or more techniques of this disclosure.

Example 1. An implantable medical device comprising: a connector block configured to couple to a plurality of leads comprising: a first lead carrying a first set of electrodes and configured to be implanted in an inter-ventricular septum of a heart to position at least one electrode of the first set of electrodes in a left bundle branch area proximate a left-ventricular septum; and a second lead carrying a second set of electrodes and configured to be implanted in a coronary sinus of the heart; and sensing circuitry configured to: sense, via the second set of electrodes, a first depolarization resulting from a first pacing pulse delivered by the first set of electrodes; and sense, via the first set of electrodes, a second depolarization resulting from a second pacing pulse delivered by the second set of electrodes; and processing circuitry configured to: determine a first conduction time of the first pacing pulse from the left-ventricular septum to the coronary sinus based on the sensing of the first depolarization; determine a second conduction time of the second pacing pulse from the coronary sinus to the left-ventricular septum based on the sensing of the second depolarization; and determine, based on the first conduction time and the second conduction time, whether the first pacing pulse comprises left bundle branch pacing.

Example 2. The implantable medical device of example 1, wherein the processing circuitry is configured to determine whether the first pacing pulse comprises left bundle branch pacing by determining whether a difference of the second conduction time minus the first conduction time is greater than or equal to a conduction time differential threshold.

Example 3. The implantable medical device of example 2, wherein the conduction time differential threshold is 20 milliseconds.

Example 4. The implantable medical device of any of examples 1 to 3, wherein the processing circuitry is further configured to: responsive to determining that the first pacing pulse comprises left bundle branch pacing, determine a respective first conduction time of a plurality of first conduction times for each atrioventricular delay of a plurality of atrioventricular delays; and select one of the atrioventricular delays based on the plurality of first conduction times.

Example 5. The implantable medical device of any of examples 1 to 4, wherein the processing circuitry is further configured to: responsive to determining the first pacing pulse does not comprise left bundle branch pacing, determine whether the first conduction time is greater than or equal to a first conduction time threshold; and responsive to determining that the first conduction time is greater than or equal to the first conduction time threshold, control delivery of cardiac resynchronization therapy via the first set of electrodes and the second set of electrodes.

Example 6. The implantable medical device of examples 5, wherein the cardiac resynchronization therapy comprises delaying a pacing pulse delivered via the second set of electrodes relative to a pacing pulse delivered via the first set of electrodes by a left-ventricular pacing pulse delay length.

Example 7. The implantable medical device of example 6, wherein the processing circuitry is configured to determine the left-ventricular pacing pulse delay length based on an equation 1.5*y−T₁=z, wherein y is a reference conduction time from the left-ventricular septum to the coronary sinus, wherein T₁ is the first conduction time, and wherein z is the left-ventricular pacing pulse delay length.

Example 8. The implantable medical device of example 7, wherein the reference conduction time is about 85 milliseconds.

Example 9. The implantable medical device of any of examples 5 to 8, wherein the first conduction time threshold is 85 milliseconds.

Example 10. The implantable medical device of any of examples 5 to 9, wherein the processing circuitry is further configured to: responsive to determining that the first conduction time is less than the first conduction time threshold, determine a respective first conduction time of a plurality of first conduction times for each atrioventricular delay of a plurality of atrioventricular delays; and select one of the atrioventricular delays based on the plurality of first conduction times.

Example 11. A medical system comprising: a plurality of leads comprising: a first lead carrying a first set of electrodes and configured to be implanted in an inter-ventricular septum of a heart to position at least one electrode of the first set of electrodes in a left bundle branch area proximate a left-ventricular septum; and a second lead carrying a second set of electrodes and configured to be implanted in a coronary sinus of the heart; an implantable medical device coupled to the plurality of leads, the implantable medical device comprising: sensing circuitry configured to: sense, via the second set of electrodes, a first depolarization resulting from a first pacing pulse delivered by the first set of electrodes; and sense, via the first set of electrodes, a second depolarization resulting from a second pacing pulse delivered by the second set of electrodes; and processing circuitry configured to: determine a first conduction time of the first pacing pulse from the left-ventricular septum to the coronary sinus based on the sensing of the first depolarization; determine a second conduction time of the second pacing pulse from the coronary sinus to the left-ventricular septum based on the sensing of the second depolarization; and determine, based on the first conduction time and the second conduction time, whether the first pacing pulse comprises left bundle branch pacing; and an external device configured to: communicatively couple to the implantable medical device; and output an indication of whether the first pacing pulse comprises left bundle branch pacing.

Example 12. The medical system of example 11, wherein the processing circuitry is configured to determine whether the first pacing pulse comprises left bundle branch pacing by determining whether a difference of the second conduction time minus the first conduction time is greater than or equal to a conduction time differential threshold.

Example 13. The medical system of example 12, wherein the conduction time differential threshold is 20 milliseconds.

Example 14. The medical system of any of examples 11 to 13, wherein the processing circuitry is further configured to: responsive to determining that the first pacing pulse comprises left bundle branch pacing, determine a respective first conduction time of a plurality of first conduction times for each atrioventricular delay of a plurality of atrioventricular delays; and select one of the atrioventricular delays based on the plurality of first conduction times.

Example 15. The medical system of any of examples 11 to 14, wherein the processing circuitry is further configured to: responsive to determining the first pacing pulse does not comprise left bundle branch pacing, determine whether the first conduction time is greater than or equal to a first conduction time threshold; and responsive to determining that the first conduction time is greater than or equal to the first conduction time threshold, control delivery of cardiac resynchronization therapy via the first set of electrodes and the second set of electrodes.

Example 16. The medical system of example 15, wherein the cardiac resynchronization therapy comprises delaying a pacing pulse delivered via the second set of electrodes relative to a pacing pulse delivered via the first set of electrodes by a left-ventricular pacing pulse delay length.

Example 17. The medical system of example 16, wherein the processing circuitry is configured to determine the left-ventricular pacing pulse delay length based on an equation 1.5*y−T₁=z, wherein y is a reference conduction time from the left-ventricular septum to the coronary sinus, wherein T₁ is the first conduction time, and wherein z is the left-ventricular pacing pulse delay length.

Example 18. The medical system of example 17, wherein the reference conduction time is about 85 milliseconds.

Example 19. The medical system of any of examples 15 to 18, wherein the first conduction time threshold is 85 milliseconds.

Example 20. The medical system of any of examples 15 to 19, wherein the processing circuitry is further configured to: responsive to determining that the first conduction time is less than the first conduction time threshold, determine a respective first conduction time of a plurality of first conduction times for each atrioventricular delay of a plurality of atrioventricular delays; and select one of the atrioventricular delays based on the plurality of first conduction times.

Example 21. A method comprising: sensing, via a second set of electrodes of a second lead of an implantable medical device, a first depolarization resulting from a first pacing pulse delivered by a first set of electrodes of a first lead of the implantable medical device, wherein the second set of electrodes is positioned in the coronary sinus, wherein the first set of electrodes is implanted in an inter-ventricular septum of a heart, and wherein at least one electrode of the first set of electrodes in a left bundle branch area proximate a left-ventricular septum; and sensing, via the first set of electrodes, a second depolarization resulting from a second pacing pulse delivered by the second set of electrodes; determining, by processing circuitry of the implantable medical device, a first conduction time of the first pacing pulse from the left-ventricular septum to the coronary sinus based on the sensing of the first depolarization; determining, by the processing circuitry, a second conduction time of the second pacing pulse from the coronary sinus to the left-ventricular septum based on the sensing of the second depolarization; and determining, by the processing circuitry, based on the first conduction time and the second conduction time, whether the first pacing pulse comprises left bundle branch pacing.

Example 22. The method of example 21, wherein determining whether the first pacing pulse comprises left bundle branch pacing comprises determining, by the processing circuitry, whether a difference of the second conduction time minus the first conduction time is greater than or equal to a conduction time differential threshold.

Example 23. The method of example 22, wherein the conduction time differential threshold is 20 milliseconds.

Example 24. The method of any of examples 21 to 23, further comprising: responsive to determining that the first pacing pulse comprises left bundle branch pacing, determining, by the processing circuitry, a respective first conduction time of a plurality of first conduction times for each atrioventricular delay of a plurality of atrioventricular delays; and selecting, by the processing circuitry, one of the atrioventricular delays based on the plurality of first conduction times.

Example 25. The method of any of examples 21 to 24, further comprising: responsive to determining the first pacing pulse does not comprise left bundle branch pacing, determining, by the processing circuitry, whether the first conduction time is greater than or equal to a first conduction time threshold; and responsive to determining that the first conduction time is greater than or equal to the first conduction time threshold, controlling, by the processing circuitry, delivery of cardiac resynchronization therapy via the first set of electrodes and the second set of electrodes.

Example 26. The method of example 25, wherein the cardiac resynchronization therapy comprises delaying a pacing pulse delivered via the second set of electrodes relative to a pacing pulse delivered via the first set of electrodes by a left-ventricular pacing pulse delay length.

Example 27. The method of example 26, wherein determining the left-ventricular pacing pulse delay length comprises determining, by the processing circuitry, the left-ventricular pacing pulse delay length based on an equation 1.5*y−T₁=z, wherein y is a reference conduction time from the left-ventricular septum to the coronary sinus, wherein T₁ is the first conduction time, and wherein z is the left-ventricular pacing pulse delay length.

Example 28. The method of example 27, wherein the reference conduction time is about 85 milliseconds.

Example 29. The method of any of examples 25 to 28, wherein the first conduction time threshold is 85 milliseconds.

Example 30. The method of any of examples 25 to 29, further comprising: responsive to determining that the first conduction time is less than the first conduction time threshold, determining, by the processing circuitry, a respective first conduction time of a plurality of first conduction times for each atrioventricular delay of a plurality of atrioventricular delays; and selecting, by the processing circuitry one of the atrioventricular delays based on the plurality of first conduction times.

1. An implantable medical device comprising:
a connector block configured to couple to a plurality of leads comprising:
a first lead carrying a first set of electrodes and configured to be implanted in an inter-ventricular septum of a heart to position at least one electrode of the first set of electrodes in a left bundle branch area proximate a left-ventricular septum; and
a second lead carrying a second set of electrodes and configured to be implanted in a coronary sinus of the heart; and
sensing circuitry configured to:
sense, via the second set of electrodes, a first depolarization resulting from a first pacing pulse delivered by the first set of electrodes; and
sense, via the first set of electrodes, a second depolarization resulting from a second pacing pulse delivered by the second set of electrodes; and
processing circuitry configured to:
determine a first conduction time of the first pacing pulse from the left-ventricular septum to the coronary sinus based on the sensing of the first depolarization;
determine a second conduction time of the second pacing pulse from the coronary sinus to the left-ventricular septum based on the sensing of the second depolarization; and
determine, based on the first conduction time and the second conduction time, whether the first pacing pulse comprises left bundle branch pacing.

2. The implantable medical device of claim 1, wherein the processing circuitry is configured to determine whether the first pacing pulse comprises left bundle branch pacing by determining whether a difference of the second conduction time minus the first conduction time is greater than or equal to a conduction time differential threshold.

3. The implantable medical device of claim 2, wherein the conduction time differential threshold is 20 milliseconds.

4. The implantable medical device of claim 1, wherein the processing circuitry is further configured to:
responsive to determining that the first pacing pulse comprises left bundle branch pacing, determine a respective first conduction time of a plurality of first conduction times for each atrioventricular delay of a plurality of atrioventricular delays; and
select one of the atrioventricular delays based on the plurality of first conduction times.

5. The implantable medical device of claim 1, wherein the processing circuitry is further configured to:
responsive to determining the first pacing pulse does not comprise left bundle branch pacing, determine whether the first conduction time is greater than or equal to a first conduction time threshold; and
responsive to determining that the first conduction time is greater than or equal to the first conduction time threshold, control delivery of cardiac resynchronization therapy via the first set of electrodes and the second set of electrodes.

6. The implantable medical device of claim 5, wherein the cardiac resynchronization therapy comprises delaying a pacing pulse delivered via the second set of electrodes relative to a pacing pulse delivered via the first set of electrodes by a left-ventricular pacing pulse delay length.

7. The implantable medical device of claim 6, wherein the processing circuitry is configured to determine the left-ventricular pacing pulse delay length based on an equation 1.5*y−T₁=z, wherein y is a reference conduction time from the left-ventricular septum to the coronary sinus, wherein T₁ is the first conduction time, and wherein z is the left-ventricular pacing pulse delay length.

8. The implantable medical device of claim 7, wherein the reference conduction time is about 85 milliseconds.

9. The implantable medical device of claim 5, wherein the first conduction time threshold is 85 milliseconds.

10. The implantable medical device of claim 5, wherein the processing circuitry is further configured to:
responsive to determining that the first conduction time is less than the first conduction time threshold, determine a respective first conduction time of a plurality of first conduction times for each atrioventricular delay of a plurality of atrioventricular delays; and
select one of the atrioventricular delays based on the plurality of first conduction times.

11. A medical system comprising:
the implantable medical device of claim 1;
a plurality of leads comprising the first lead and the second lead, the implantable medical device coupled to the plurality of leads; and
an external device configured to:
communicatively couple to the implantable medical device; and
output an indication of whether the first pacing pulse comprises left bundle branch pacing.

12. A method comprising:
sensing, via a second set of electrodes of a second lead of an implantable medical device, a first depolarization resulting from a first pacing pulse delivered by a first set of electrodes of a first lead of the implantable medical device, wherein the second set of electrodes is positioned in the coronary sinus, wherein the first set of electrodes is implanted in an inter-ventricular septum of a heart, and wherein at least one electrode of the first set of electrodes in a left bundle branch area proximate a left-ventricular septum; and
sensing, via the first set of electrodes, a second depolarization resulting from a second pacing pulse delivered by the second set of electrodes;
determining, by processing circuitry of the implantable medical device, a first conduction time of the first pacing pulse from the left-ventricular septum to the coronary sinus based on the sensing of the first depolarization;
determining, by the processing circuitry, a second conduction time of the second pacing pulse from the coronary sinus to the left-ventricular septum based on the sensing of the second depolarization; and
determining, by the processing circuitry, based on the first conduction time and the second conduction time, whether the first pacing pulse comprises left bundle branch pacing.

13. The method of claim 12, wherein determining whether the first pacing pulse comprises left bundle branch pacing comprises determining, by the processing circuitry, whether a difference of the second conduction time minus the first conduction time is greater than or equal to a conduction time differential threshold.

14. The method of claim 13, wherein the conduction time differential threshold is 20 milliseconds.

15. The method of claim 12, further comprising:
responsive to determining that the first pacing pulse comprises left bundle branch pacing, determining, by the processing circuitry, a respective first conduction time of a plurality of first conduction times for each atrioventricular delay of a plurality of atrioventricular delays; and
selecting, by the processing circuitry, one of the atrioventricular delays based on the plurality of first conduction times.

16. The method of claim 12, further comprising:
responsive to determining the first pacing pulse does not comprise left bundle branch pacing, determining, by the processing circuitry, whether the first conduction time is greater than or equal to a first conduction time threshold; and
responsive to determining that the first conduction time is greater than or equal to the first conduction time threshold, controlling, by the processing circuitry, delivery of cardiac resynchronization therapy via the first set of electrodes and the second set of electrodes.

17. The method of claim 12, further comprising:
responsive to determining the first pacing pulse does not comprise left bundle branch pacing, determining, by the processing circuitry, whether the first conduction time is greater than or equal to a first conduction time threshold; and
responsive to determining that the first conduction time is greater than or equal to the first conduction time threshold, controlling, by the processing circuitry, delivery of cardiac resynchronization therapy via the first set of electrodes and the second set of electrodes.

18. The method of claim 17, wherein the cardiac resynchronization therapy comprises delaying a pacing pulse delivered via the second set of electrodes relative to a pacing pulse delivered via the first set of electrodes by a left-ventricular pacing pulse delay length.

19. The method of claim 18, further comprising:
determining, by the processing circuitry, the left-ventricular pacing pulse delay length based on an equation 1.5*y−T₁=z, wherein y is a reference conduction time from the left-ventricular septum to the coronary sinus, wherein T₁ is the first conduction time, and wherein z is the left-ventricular pacing pulse delay length.

20. The method of claim 17, further comprising:
responsive to determining that the first conduction time is less than the first conduction time threshold, determining, by the processing circuitry, a respective first conduction time of a plurality of first conduction times for each atrioventricular delay of a plurality of atrioventricular delays; and
selecting, by the processing circuitry one of the atrioventricular delays based on the plurality of first conduction times.
